(19) United States
(12) Patent Application Publication
(10) Pub. No.: US 2006/0105962 A1
(43) Pub. Date: May 18, 2006

(54) TONIC TO MAINTAIN INTESTINAL FLORA

(76) Inventor: Robert Owen Alexander Robinson, Kingston (JM)

Correspondence Address: MALIN HALEY AND DIMAGGIO, PA, 1936 S ANDREWS AVENUE, FORT LAUDERDALE, FL, 33316, US

(21) Appl. No.: 11/160,711

(22) Filed: Jul. 6, 2005

(30) Foreign Application Priority Data
Nov. 14, 2004 (JM) 18/14333

(51) Int. Cl. A61K 31/7024; A61K 31/70; A61K 31/19

(52) U.S. Cl. 514/23; 514/557

(57) ABSTRACT

An orally-administered pre-biotic composition for promoting the growth of normal, healthy intestinal flora while preventing the proliferation of unhealthy and toxic intestinal flora. When ingested, the composition improves the natural biological protection of the human body against toxic substances that are present in foods ingested and those that are produced by abnormal intestinal flora. The suspension is comprised of a source of fructose, esters of short chain fatty acids, salts of short chain fatty acids, sodium or potassium chloride, monocalcium phosphate, and sodium citrate.

### FIELD OF THE INVENTION

This invention relates to an orally-administered, pre-biotic composition for normalizing and maintaining the normal, healthy intestinal flora of the human body by protecting against the effects of toxic substances that are present in foods as well as toxins produced by abnormal intestinal flora.

### DESCRIPTION OF RELATED ART

Poor biological health of the intestine, and particularly of the large intestine, increases the possibility that undigested products may be absorbed into the body before being detoxified by microbial action. Abnormal and toxic microbial flora may proliferate in an unhealthy intestinal environment. These microorganisms may produce harmful metabolites or may release antigenic products that are subsequently absorbed, thereby resulting in direct toxic or immunological effects on the body. The effects of these undesirable, absorbed products may be summarized under two broad categories—direct toxicity and indirect toxicity.

Direct toxicity involves metabolites or antigenic products absorbed by the body directly or by secondary immunological reaction, which cause clinical disease. For example, a vasoactive compound, when absorbed by the body, causes changes in the blood vessel size in brain tissues resulting in a migraine or an unexplained headache. Moreover, some absorbed products have antigenic properties that react with the human body's antigens leading to an immunological reaction that causes an arthritic process.

Indirect toxicity refers to an absorbed product that affects immune cell function or requires significant immunological activity to contain its presence. These indirectly toxic metabolites and antigenic products, which are produced by abnormal intestinal flora, may cause a relative immune deficiency state that compromises the body's efficiency in disposing of concurrent conditions. For example, a person develops a persistent sinusitis or recurrent asthmatic condition because the individual's body is unable to mount an adequate immune response to resolve the condition.

Natural honey consists of 80% sugars, most of which are monosaccarides, especially fructose. In addition, honey contains many valuable nutritional components including vitamins and minerals. Due to its osmotic state, acidity, and the presence of anti-microbial agents such as furfural and hydroxyl-methyl-furfural, honey inhibits the growth of a majority of microbial agents. Natural honey also contains small amounts of valeric acid, and rarely, caproic acid, which also have anti-microbial properties. Valeric acid and caproic acid are short chain fatty acids, C5 and C6 respectively. These acids are produced by normal intestinal flora, and thus, are normally present in the intestines. The intestines normally contain valeric acid in a range of 2.0-4.0 mmol/kg of the colonic contents and caproic acid in a range of 1.0-2.0 mmol/kg of the colonic contents. The production of these short chain fatty acids by bacteria present in the normal flora of the intestines serves to inhibit the growth of microorganisms, such as yeast, that may contribute to the development of an unhealthy intestinal flora. These microorganisms compete with healthy intestinal flora for colonization of the intestinal space. The inhibitory effect exhibited by valeric and caproic acids with regards to unhealthy intestinal flora is due primarily to the fatty acid moiety of these acids. The acidic component, or the hydrogen ions, may also play a minor role in the inhibitory process. The potency of these short chain fatty acids in inhibiting the growth of unhealthy intestinal flora is greater with increasing numbers of carbon atoms in the short chain fatty acid molecule.

Honey also contains two other beneficial compounds, furfural and hydroxy-methyl-furfural, which are natural compounds found in honey that exhibit both antibacterial and antifungal properties. The primary effect of furfural compounds is observed in yeast, where furfural significantly reduces the numbers and functional capacity of these organisms. Furfural compounds are absorbed readily by the small intestine, and thus, influence the microbial flora of the gut proximal to the large intestine. Heating honey increases the presence of these compounds through a process known as the Miallard reaction. After heating honey, total furfural content ranges between 40-250 mg/kg of honey depending on the source of said honey. Studies with rats have demonstrated up to 100% safety with exposure to furfural compounds of 53.0 mg/kg body weight over a thirteen week period.

Carboxylic acids such as valeric acid and caproic acid react with sugars, such as fructose and glucose, which are present in honey, to form esters. The rate of these reactions is increased with increasing temperature. Esters of carboxylic acids, or short chain fatty acids (SCFAs), especially those formed with fructose, are poorly digested and absorbed by the small intestine. A significant portion of the undigested esters bypass the small intestine and enter the large intestine where normal bacterial flora hydrolyse the esters. These organisms, e.g. Lactobacillus acidophilus, consume and proliferate by consuming the sugar component of the esters and releasing the carboxylic acid component. The release of valeric acid and caproic acid further inhibits the growth of abnormal flora, especially the growth of yeasts and fungal organisms. The rate of digestion and absorption decreases with increases in the length of the carbon chain of the carboxylic acid. Thus, valeric esters (C5) are more digestible and absorbable than caproic esters (C6). The digestion of these esters releases the individual components of each ester molecule, i.e., carboxylic acid and sugars, e.g. fructose. Both valeric acid and caproic acid are anti-microbial and are especially effective inhibitors of the growth of yeast and other fungal organisms. Caproic acid is a stronger inhibitor than valeric acid. Digestion of esters in the small intestine results in a sequential inhibition of microbes, especially yeasts and fungal organisms, starting with the inhibitory effects of valeric acid and augmented later by the effects of caproic acid.

### SUMMARY OF THE INVENTION

The applicant's invention is for an ingestible pre-biotic composition, useful in improving the biological, protective health of the intestine, and primarily, of the large intestine. The composition comprises a source of fructose, esters of short chain fatty acids, salts of short chain fatty acids, sodium or potassium chloride, monocalcium phosphate, and sodium citrate.

Short chain fatty acids are aliphatic carboxylic acids, which are weak organic acids containing the carboxyl group (R) COOH. In this case, (R) refers to the number of carbon atoms present, which range from 0 to 5 and includes isomers of the acids.

Fructose is available from several carbohydrate sources, including, but not limited to, honey and fruits, in which fructose occurs naturally, as well as commercially-produced fructose created through the processing of saccharides, e.g., commercial high fructose corn syrup.

The addition of SCFA salts to the composition increases the shelf-life of the product and also aids in small intestinal control of the growth of abnormal intestinal flora. Furfural and hydroxy-furfural produced by heating the fructose solutions, especially those solutions containing natural honey, may also influence microbial flora growth in the small intestine. Monocalcium phosphate and sodium citrate are added to the composition primarily to reduce the pungent smell of valeric acid and caproic acid in the mixture.

Healthy intestinal flora is a major factor in maintaining good intestinal health in humans. Healthy intestinal flora is defined as the bacteria and other microorganisms of the small and large intestines which remove potentially harmful, undigested toxic agents from a normal diet without producing any potentially harmful, toxic byproducts during fermentation. These toxic agents include antigens and vasoactive metabolites, among others, produced by unhealthy intestinal flora, which may interfere with the normal physiological function of the body directly or indirectly, thereby resulting in health problems. The toxic agents further include undigested products present in a normal human diet that may have similar harmful effects. Through fermentation, healthy intestinal flora produces substances that may specifically or non-specifically enhance the human body's immune system. Poor biological health of the intestines results in decreased production of immune enhancers by this flora. These immune enhancers, which improve the function of the human immune system, promote the effectiveness of the body's immune system in controlling and/or eliminating diseases. Poor intestinal health also increases the possibility of undigested, harmful substances being absorbed into the body, before being detoxified by microbial action. In addition, unhealthy intestinal flora, which includes any small or large intestinal flora that is not healthy intestinal flora, may proliferate in an unhealthy intestinal environment and may produce harmful metabolites, resulting in direct toxic effects on the body. The effects of this undesirable, unhealthy intestinal flora may be summarized under two broad categories: loss of immune enhancers and absorption of toxic agents.

An unhealthy intestinal environment reduces the production and absorption of certain metabolites (immune enhancers), produced by healthy intestinal flora, which enhance the functioning of the body's immune system. These immune enhancers may be either specific or non-specific, with specific enhancers being produced on demand, possibly, by communication between white blood cells and healthy intestinal flora. Current scientific theories suggest that white blood cells expose the healthy intestinal flora to substances extracted from disease processes of the human body, e.g., cancer. Subsequent to exposure to these substances, the healthy intestinal flora, which are very adaptable, mutate to produce genes that counteract these extracts. Researchers believe that the healthy intestinal flora may transmit this mutated genetic material to the human body's white blood cells, possibly via plasmid transfer. Plasmid transfer refers to the transfer of cytoplasmic genetic material, or plasmids, from bacterial cytoplasm to white blood cells where said genetic material is incorporated into the genetic material of the white blood cells.

Possible sites for this genetic exchange may include the appendix and the cecum. These pouch-like structures could contain the extracts produced by disease processes in the body for longer periods of time, thereby allowing the body's healthy intestinal flora a better opportunity to develop resistance to said harmful substances through longer periods of exposure to said toxins. Moreover, the walls of these anatomical structures, especially the appendix, are lined with white cells that facilitate communication between the body's immune system and the healthy intestinal flora. This process of adapting to extracts produced by disease processes in the human body through repeated and lengthy exposure to said extracts represents a form of self-immunization. The absence of healthy intestinal flora may hinder the communication process described above, thereby resulting in loss of enhancement of the body's immune system as well as failure to control disease processes, e.g., certain cancers, sinusitis, asthma, and acne.

The second undesirable effect of unhealthy intestinal flora involves decreases in the absorption of toxic agents. Unhealthy intestinal flora may decrease detoxification of harmful undigested products or may, itself, produce toxic agents. Researchers theorize that these toxic agents may enter the blood and directly or indirectly interfere with physiological function. The absorption of a vasoactive metabolite, which may alter blood vessel size and result in diseases such as migraine headaches or essential hypertension, is an example of direct interference with physiological function. Toxic agents also may have antigenic properties and cross-react with the body's antigens resulting in an immune reaction and disease processes, e.g., collagen vascular diseases. Indirectly, these toxic agents may also adversely affect the body's immune system, resulting in the inability of the body to fight disease.

While unhealthy intestinal flora may produce toxins that adversely affect the human body, poor intestinal health is most likely to occur in humans due to exposure to various ingested food products having anti-bacterial properties that upset or kill healthy intestinal flora. The use of antibiotic therapy, orally or parenterally, may also be a factor. The following list names several potential sources of disturbances in the normal healthy intestinal flora of the human body.

A. Use of anti-bacterial agents directly in the treatment of diseases, e.g., usage of antibiotics to treat skin infections.

B. Use of anti-bacterial agents in the production of meat. Residues of these anti-bacterial agents may be present in animals after slaughter and in ingested meat.

C. Use of chemicals in food production. Many herbicides and pesticides used in farming activity may persist in food products after harvesting and processing. These agents often have anti-bacterial properties and may cause unhealthy intestinal flora to flourish in the human intestinal tract when eaten.

D. Foods containing preservatives, when ingested, may lead to the development of unhealthy intestinal flora.

E. Changes in the molecular structure and solubility of processed foods may create anti-bacterial properties in these foods. For example, gliadin, which is produced in high levels by the processing of gluten, is a soluble polypeptide that may have a low-grade anti-bacterial effect, thereby resulting in the development and growth of unhealthy intestinal flora.

F. Chemicals used in municipal water treatment, such as chlorine, may cause the alteration of bacterial flora within the intestines.

Valeric and caproic acids produced by bacteria in the normal flora of the intestine inhibit the growth of other microorganisms, such as yeast, which may compete for colonization of the intestinal space, and as a result, cause human health problems. The combination of furfural, a compound found in natural honey, with valeric acid and caproate esters provides an individual with a total gut inhibitory effect on abnormal bacterial flora and enhances normal bacterial flora allowing for proper intestinal biological protection, and hence, improved health.

To influence the growth of microbial flora in the human intestines through dietary supplementation of these SCFAs, the acids must be present not only in the small intestine, but more importantly, must bypass the small intestine and enter the large intestine. For this reason, esters of SCFAs, especially those formed from fructose or hexanol, are poorly digested and absorbed by the small intestine. The digestion of esters in the small intestine involves enzymatic hydrolysis of the esters. This hydrolysis is partially inhibited in the presence of free fructose molecules. The digestion of SCFAs in the small intestine releases the fatty acid moiety of said esters, thus negatively influencing the growth of unhealthy intestinal flora, including yeast, within the small intestine.

The rate of digestion and absorption of these esters decreases with increases in the length of the carbon chain of the SCFAs. Hence, acetic esters (C2) are more digestible and absorbable than caproic esters (C6). A significant portion of the undigested esters, especially valeric and caproic esters, bypass the small intestine and enter the large intestine. These esters are rapidly hydrolysed by bacterial flora, thereby causing the immediate release of the fatty acid moiety within the large intestine. Thus, dietary supplementation with SCFA esters in the presence of free fructose causes both large and small intestinal inhibition of abnormal unhealthy intestinal flora. In this way, the applicant's composition assists in removing toxic agents and encouraging the production of immune enhancers.

An object of this invention is to provide a composition that enhances and maintains the growth of normal, healthy intestinal flora to promote human health and prevent disease.

Another object of this invention is to provide a composition that inhibits and eliminates the growth of abnormal, unhealthy intestinal flora, including certain types of bacteria, yeast, and other fungal species and microorganisms.

Still another object of this invention is to provide a natural composition, which is safe for human ingestion, to promote the proliferation of normal intestinal flora while inhibiting the colonization of abnormal toxin-producing intestinal flora.

In accordance with these and other objects which will become apparent hereinafter, the instant invention will now be described with particular reference to the accompanying drawings.

### BRIEF DESCRIPTION OF THE DRAWINGS

FIG. 1 shows a flow chart summarizing the pathogenic effects of unhealthy intestinal flora in the human intestinal tract.

FIG. 2 shows a flow chart summarizing the mechanism of action of the pre-biotic composition.

### DETAILED DESCRIPTION

The present invention is for an orally-administered, pre-biotic suspension composition, useful in improving the biological, protective health of the intestines, and primarily, of the large intestine. The composition comprises a suspension mixture that includes a source of fructose, esters of short chain fatty acids, salts of short chain fatty acids, sodium or potassium chloride, monocalcium phosphate, and sodium citrate. Said composition may be produced in the form of a food supplement or as a food additive to finished foods. Supplementary forms of the composition include, but are not limited to, suspensions, gel caps, capsules, tablets, and drops. These supplementary forms may also include pro-biotics and other pre-biotics, such as oligosaccharides. As a food additive, said composition may be included in finished foods, including, but not limited to, soft drinks and juices, nutritional bars, confectioneries, cereals, and breads.

The composition may be prepared from scratch or by obtaining commercially available components and combining them in appropriate proportions as indicated in the table below. Short chain fatty acid esters, which are the major ingredients of said composition, are produced by the combination of fructose or hexanol with short chain fatty acids. Fructose esters are synthesized by heating SCFAs in a fructose solution using an acid catalyst. To produce the composition, a fructose solution, such as natural honey or high fructose corn syrup, is mixed and heated in the presence of a mixture of SFCAs and an acid catalyst, such as concentrated hydrochloric acid. The ingredients are used preferably in the proportions indicated in Table 1 below to produce said composition, although other ranges and combinations of said ingredients are possible. Combinations of SCFA esters and salts may vary, but the essential salts and esters are those of caproic acid and valeric acid. The proportions of ingredients used are described by their ratio in grams per kilogram of fructose contents.

TABLE 1

| COMPONENTS | CONTENT RANGE | |
|---|---|---|
| WATER (Varies with Type of Preparation) | 30-330 | g/kg |
| FRUCTOSE or HEXANOL ESTERS of: | | |
| Formic acid | 5-600 | mg/kg |
| Acetic acid | 10-50 | g/kg |
| Propionoic acid | 10-50 | g/kg |
| Butyric acid | 10-50 | g/kg |
| Lactic acid | 0.2-50 | g/kg |
| Valeric acid | 10-50 | g/kg |
| Caproic acid | 10-50 | g/kg |
| SCFA SALTS (Sodium or Potassium): | | |
| Acetate | 3-5 | g/kg |
| Propionate | 3-5 | g/kg |
| Butyrate | 3-5 | g/kg |
| Valerate | 3-5 | g/kg |
| Caproate | 3-5 | g/kg |
| MONOCALCIUM PHOSPHATE | 1-2 | g/kg |
| SODIUM CITRATE | 1-3 | g/kg |
| SODIUM CHLORIDE | 3-5 | g/kg |

The mixture is heated slowly (at approximately one degree Celsius per minute) to a temperature of 75 to 77 degrees Celsius. A maximum temperature of 77 degrees Celsius is used because the flavor of the honey is significantly retained up to this temperature, but the flavor is progressively lost with further increases in temperature during heating. This temperature is adequate for esterification of the SCFAs with the source of fructose, which, in this instance, is natural honey. The production of furfural and hydroxy-furfural compounds occurs when a fructose solution is heated, especially when the source of fructose used is natural honey. These compounds may augment the potency of the composition as described below. Upon reaching 77 degrees Celsius, the composition mixture is removed from the heat, and the heated mixture is allowed to cool to room temperature. Once cooled to room temperature, the mixture is agitated to homogenize the composition, and the mixture is then packaged in previously sterilized containers.

Esterification reactions are reversible, and after esterification, small amounts of free acids will be present in the mixture. These acids are volatile, having pungent odors, and may cause negative behavioral responses by users of the invention. By adding mineral acids, which are weaker than the SCFAs, such as monocalcium phosphate and sodium citrate, the level of free volatile acids is reduced, thereby preventing such odor production. Sodium or potassium salts of SCFAs are added to the composition to enhance the efficacy and shelf-life of the product.

The production of valeric and caproic acids by bacteria in the normal flora of the intestine serves to inhibit the growth of other microorganisms, such as yeast, which may compete for colonization of the intestinal space. These competing microorganisms, if allowed to grow, will produce an abnormal intestinal flora, and hence, may predispose an individual to health risks. Heating natural honey in the presence of valeric acid and caproic acid with added water increases the contact between the sugars of the honey and these acids and results in the formation of fructose carboxylic acid esters, which are essential to the efficacy of this orally-administered pre-biotic suspension composition.

The potency of short chain fatty acids in inhibiting the growth of unhealthy intestinal flora is greater with increasing numbers of carbon atoms in the short chain fatty acid molecule. Therefore, caproic acid (C6) and valeric acid (C5) are the two most potent, the former being more potent than the latter, and preferably used in the production of this composition. Likewise, formic acid (C1) and acetic acid (C2) are the two least potent SCFAs, and therefore, preferably are not used to make the composition.

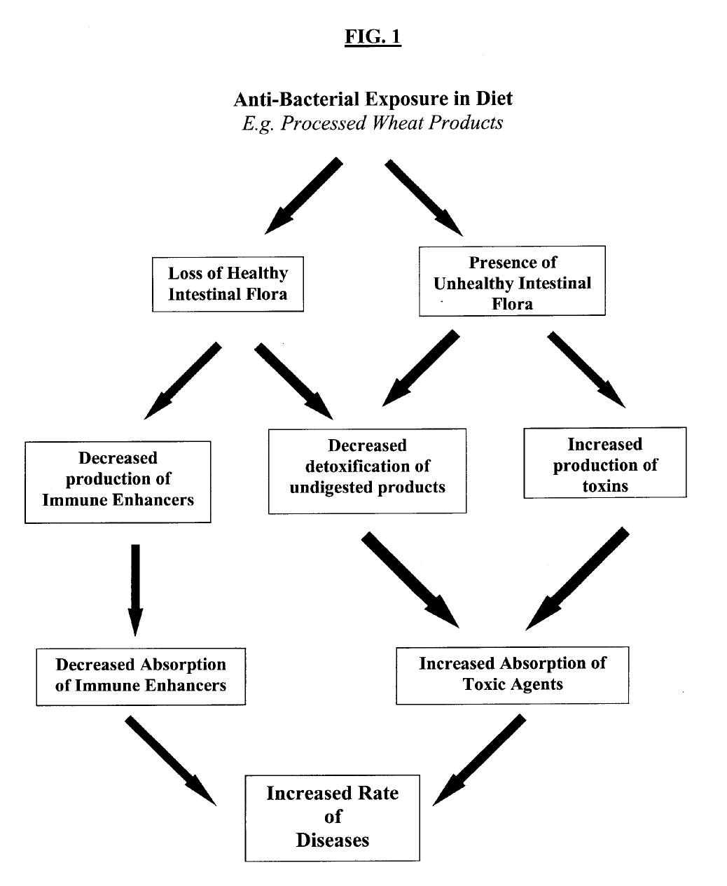

The combination of furfural with valeric acid and caproate esters provides an individual with a total gut inhibitory effect on abnormal bacterial flora and enhances normal bacterial flora allowing for proper intestinal biological protection, and hence, improved health. FIG. 1 illustrates a flow chart summarizing the pathogenic effects of unhealthy intestinal flora in the human intestinal tract. The intake amount of furfural compounds when ingesting this pre-biotic suspension would range between 0.2-2.5 mg daily at a dose of one-half to one teaspoon twice daily. Using the above-prescribed dosage as the maximum daily intake of furfural compounds for an individual equates to less than 0.5 mg/kg body weight, which is considered 100% safe for humans.

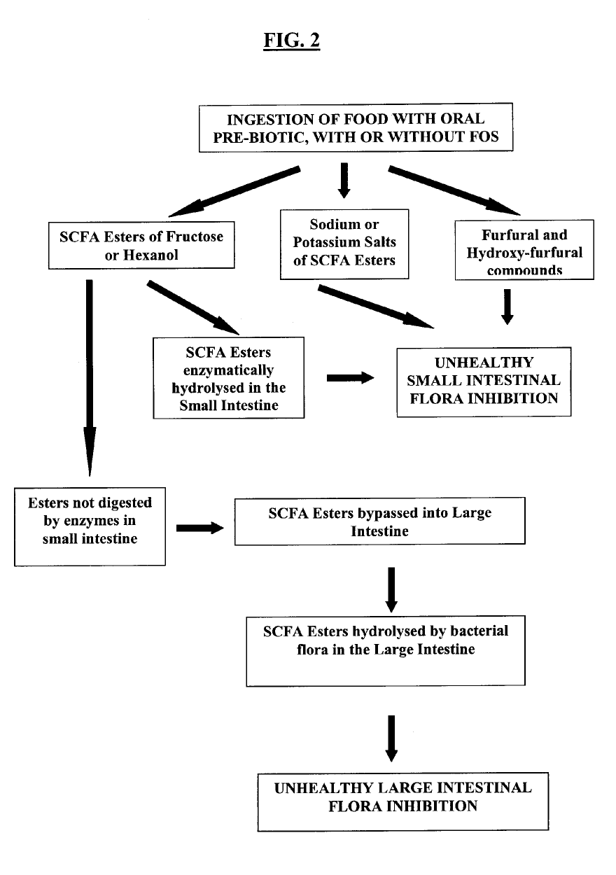

FIG. 2 shows a flow chart summarizing the mechanism of action of the pre-biotic composition. To maintain normal healthy intestinal flora, an individual should ingest 2.5 milliliters of the composition two or three times daily if said individual weighs less than 20 kg and 5.0 milliliters of the composition two or three times daily if said individual weighs more than 20 kg. When ingested with meals, the composition ensures the proliferation of healthy microbial flora within the human intestinal tract. This proliferative process is potentiated by the addition of highly nutritious, undigestible food components, such as fructo-oligosaccharides (FOS). Generally, fructo-oligosaccharides are added to the composition in a range of 0.2 to 2.0 parts FOS to 1.0 part fructose. Pro-biotics, which contain organisms that are part of a healthy intestinal flora, may also be added to further enhance this process. Either FOS, pro-biotics, or a combination of the two may be added to the pre-biotic composition in the form of a suspension, capsule or tablet.

The instant invention has been shown and described herein in what is considered to be the most practical and preferred embodiment. The applicant recognizes, however, that departures may be made therefrom within the scope of the invention and that obvious modifications will occur to a person skilled in the art.

What is claimed is:

1. A composition for re-establishing and maintaining normal intestinal flora, thus optimizing microbial protection within the gut, said composition comprising a mixture of the following:
a source of fructose;
esters of short chain fatty acids;
salts of short chain fatty acids;
sodium or potassium chloride;
monocalcium phosphate for preventing pungent odor produced by free acids after esterification; and
sodium citrate for preventing pungent odor produced by free acids after esterification.

2. The composition of claim 1, wherein the short chain fatty acids, esters of which are included in the composition, comprise the following:
formic acid;
acetic acid;
propionoic acid;
lactic acid;
butyric acid;
valeric acid; and
caproic acid.

3. The composition of claim 1, wherein said salts may include either sodium or potassium salts of the following short chain fatty acids for enhancing the efficacy and shelf-life of the composition:
acetic acid;
propionoic acid;
butyric acid;
valeric acid; and
caproic acid.

4. The composition of claim 1, wherein valeric acid and caproic acid are the preferred short chain fatty acids included in said composition.

5. The composition of claim 1, wherein natural honey and high fructose corn syrup are the preferred sources of fructose included in said composition with natural honey being the most preferred source of fructose.

6. The composition of claim 1, wherein said composition may be produced as a suspension, gel cap, capsule, or tablet, and may also be added to solid matrix foods, such as nutrition bars and cereals, and to nutritional supplements, such as fruit drinks and liquid meal supplements.

7. The composition of claim 1, wherein said composition may include fructo-oligosaccharides, pro-biotics, or a combination of both, which may be added to the composition.

8. The composition of claim 1, wherein said composition comprises a mixture of the following ingredients in the following preferred amounts measured in ratios of mass per kilogram of fructose content:
water (the amount varies with type of preparation), 30.0-330.0 g/kg;
a combination of fructose esters, comprised of fructose and short chain fatty acids, chosen from among the following:
formic acid, 5.0-600.0 mg/kg;
acetic acid, 10.0-50.0 g/kg;
propionoic acid, 10.0-50.0 g/kg;
butyric acid, 10.0-50.0 g/kg;
lactic acid, 0.2-50.0 g/kg;
valeric acid, 10.0-50.0 g/kg;
caproic acid, 10.0-50.0 g/kg;
a combination of short chain fatty acid (sodium or potassium) salts chosen from among the following:
acetate, 3.0-5.0 g/kg;
propionate, 3.0-5.0 g/kg;
butyrate, 3.0-5.0 g/kg;
valerate, 3.0-5.0 g/kg;
caproate, 3.0-5.0 g/kg;
monocalcium phosphate, 1.0-2.0 g/kg;
sodium citrate, 1.0-3.0 g/kg; and
sodium chloride, 3.0-5.0 g/kg.

9. The composition of claim 1, wherein by re-establishing and maintaining normal intestinal flora, said composition reduces and removes various toxic substances that cause diseases directly or through immunological reactions.

10. The composition of claim 1, wherein by re-establishing and maintaining normal intestinal flora, said composition improves the human body's immunological responses, thereby enhancing the healing process for diseases generally.

11. The composition of claim 1, wherein said composition is a dietary source of important vitamins and mineral supplements.

12. The composition of claim 1, wherein said composition contains only naturally occurring chemicals.

13. The composition of claim 1, wherein said composition is preferably orally administered as a pre-biotic suspension.

14. The composition of claim 1, wherein to maintain normal healthy human intestinal flora, said composition is ingested twice daily by an individual in the amount of 2.5 milliliters if said individual weighs less than 20 kg and 5.0 milliliters if said individual weighs more than 20 kg.

15. A method for re-establishing and maintaining normal human intestinal flora, thus optimizing microbial protection from colonization by harmful toxin-producing bacteria, fungi, and other microorganisms within the human gut, said method comprising the following steps:
preparing a composition comprised of a mixture of the following ingredients:
a source of fructose;
esters of short chain fatty acids;
salts of short chain fatty acids;
sodium or potassium chloride;
monocalcium phosphate; and
sodium citrate; and
ingesting said mixture in predetermined doses at predetermined intervals of time.

16. The method of claim 15, wherein natural honey is the preferred source of fructose used in said composition.

17. The method of claim 15, wherein esters of valeric acid and caproic acid are the preferred esters of short chain fatty acids used in said composition.

18. The method of claim 15, wherein said composition mixture is prepared by the following steps:
mixing a source of fructose, esters of short chain fatty acids, salts of short chain fatty acids, and sodium chloride or potassium chloride in a container;
heating said mixture slowly;
removing said mixture from the heat and adding monocalcium phosphate and sodium citrate;
allowing said mixture to cool to room temperature; and
agitating said mixture to homogenize the composition before packaging said mixture in previously sterilized containers.

19. The method of claim 15, wherein said mixture is heated slowly at approximately one degree Celsius per minute to a temperature equal to and no greater than 77 degrees Celsius to ensure that flavor is preserved.

20. The method of claim 15, wherein one purpose of heating said mixture is to increase the production of furfural and hydroxyl-methyl-furfural, two naturally-occurring beneficial antibacterial and antifungal compounds found in natural honey, which are absorbed readily by the small intestine, and thus, influence the growth of normal human intestinal flora in the human gut proximal to the large intestine.
